(19) United States
(12) Patent Application Publication
(10) Pub. No.: US 2002/0103445 A1
(43) Pub. Date: Aug. 1, 2002

(54) Thermography catheter with flexible circuit temperature sensors

(76) Inventors: David A. Rahdert, San Francisco, CA (US); Michael Perry, Los Altos, CA (US); Brett A. Herscher, Cupertino, CA (US); Joseph Fjelstad, Maple Valley, WA (US); Thomas H. Campbell, Brentwood, CA (US)

Correspondence Address: OPPENHEIMER WOLFF & DONNELLY LLP, 840 NEWPORT CENTER DRIVE, SUITE 700, NEWPORT BEACH, CA, 92660, US

(21) Appl. No.: 09/938,963

(22) Filed: Aug. 24, 2001

Related U.S. Application Data
(60) Provisional application No. 60/227,713, filed on Aug. 24, 2000.

(51) Int. Cl. A61B 5/00

(52) U.S. Cl. 600/549

(57) ABSTRACT

The present invention relates, generally, to thermography catheters and, more particularly, to thermography catheters which use flex circuit technology to create the connections and thermocouples used to detect hot spots (areas with high metabolic activity) of the atherosclerotic plaque, vascular lesions, and aneurysms in human vessels.

### CROSS REFERENCE TO RELATED APPLICATIONS

This application claims the benefit of U.S. Provisional Application No. 60/227,713, filed Aug. 24, 2000, whose entire contents are hereby incorporated by reference as if fully set forth herein. In addition, this application discloses subject matter related to U.S. patent application Ser. No. 09/340,089, filed on Jul. 25, 1999, naming Cassells et al. first inventor, U.S. Pat. No. 5,871,449, issued to Brown, U.S. Pat. No. 5,935,075, issued to Cassells et al., U.S. Pat. No. 5,924,997 issued to Campbell, and U.S. Pat. No. 6,245,026 issued to Campbell et al. The disclosures of the aforementioned United States patents and patent applications, are hereby incorporated herein by reference as if fully set forth herein.

### BACKGROUND OF THE INVENTION

The present invention relates, generally, to thermography catheters and, more particularly, to thermography catheters which use flex circuit technology to create the connections and thermocouples used to detect hot spots (areas with high metabolic activity) of the atherosclerotic plaque, vascular lesions, and aneurysms in human vessels.

### SUMMARY OF THE INVENTION

Cardiovascular disease is one of the leading causes of death worldwide. For example, some recent studies have suggested that plaque rupture may trigger 60 to 70% of fatal myocardial infarctions. In a further 25 to 30% of fatal infarctions, plaque erosion or ulceration is the trigger. Vulnerable plaques are often undetectable using conventional techniques such as angiography. Indeed, the majority of vulnerable plaques that lead to infarction occur in coronary arteries that appeared normal or only mildly stenotic on angiograms performed prior to the infarction.

Studies into the composition of vulnerable plaque suggest that the presence of inflammatory cells (and particularly a large lipid core with associated inflammatory cells) is the most powerful predictor of ulceration and/or imminent plaque rupture. For example, in plaque erosion, the endothelium beneath the thrombus is replaced by or interspersed with inflammatory cells. Recent literature has suggested that the presence of inflammatory cells within vulnerable plaque and thus the vulnerable plaque itself might be identifiable by detecting heat associated with the metabolic activity of these inflammatory cells. Specifically, it is generally known that activated inflammatory cells have a heat signature that is slightly above that of connective tissue cells. Accordingly, it is believed that one way to detect whether specific plaque is vulnerable to rupture and/or ulceration is to measure the temperature of the plaque walls of arteries in the region of the plaque.

Once vulnerable plaque is identified, the expectation is that in many cases it may be treated. Since currently there are not satisfactory devices for identifying and locating vulnerable plaque, current treatments tend to be general in nature. For example, low cholesterol diets are often recommended to lower serum cholesterol (i.e. cholesterol in the blood). Other approaches utilize systemic anti-inflammatory drugs such as aspirin and non-steroidal drugs to reduce inflammation and thrombosis. However, it is believed that if vulnerable plaque can be reliably detected, localized treatments may be developed to specifically address the problems.

Recently there have been several efforts to develop thermography catheters that are capable of thermally mapping vascular vessels to identify thermal hot spots that are indicative of vulnerable plaque. By way of example, commonly assigned U.S. Pat. No. 6,245,026 issued to Campbell et al. describes a number of thermography devices and combined thermography and drug delivery and/or sampling catheters. Other thermography catheters are described in U.S. Pat. No. 5,871,449 (to Brown), U.S. Pat. No. 5,935,075 (Cassells et al.) and U.S. Pat. No. 5,924,997 (Campbell), each of which are incorporated herein by reference.

Recent experiments have shown that thermography is indeed capable of thermally mapping a vessel to the degree necessary to identify vulnerable plaque. However for thermography to become popular, it is going to be critical to develop localized treatments that can be administered when vulnerable plaque is identified.

Flex circuit technology, also known as “flexible printed wiring” or “flex print”, is already established as a way to create many parallel wires in a tiny space and is used in applications where compactness and flexibility are required. Flex circuit technology is currently used in the manufacture of hearing aids, ultrasonic probe heads, cardiac pacemakers and defibrillators. Flex circuits are differentiated by their application. Static flex circuits are manipulated for installation or fit only. In contrast, dynamic flex circuits are designed to operate continuous or intermittently.

The current invention describes designs and construction techniques used to produce an interventional device that utilizes flex circuits to create a multiplicity of conductive pathways which are routed through an expandable member, for example, an intravascular balloon catheter or an expandable wire basket, creating a thermal sensor at their distal terminal point, which is adhered or mounted on the expandable member. Additionally, the current invention will describe the means by which these thermal sensors display, collect, and store its data in a control box connected to the proximal end of the interventional device.

By way of example, in a first embodiment of the invention a sheet of polyamide approximately 3 mil thick is imprinted electrochemically with conductive metallic strips approximately 0.5 mil thick and 5 mil wide spaced on a 10 mil interval to form a flex circuit. The 10-mil pattern may be repeated as many times as necessary to create a multiplicity of parallel wires depending on the needs of a particular catheter. The metal strips are electrically conductive and serve as “wires”. A single flex strip 0.25″ wide may thus contain 25 “wires”.

It will become apparent to those skilled in the art that applying this technology to a catheter having an expandable member used to detect vulnerable plaque allows for the construction of a device with enhanced flexibility and decreased profile. Various construction techniques can be utilized to create thermal sensor circuits (TSC) that operate in a range from 20 to 80 ohms, based on the particular needs of a specific catheter.

In a second embodiment of the invention, the TSC's themselves are single sided flex circuits where a single conductor layer of either metal or conductive polymer is applied to a compliant dielectric film with sensor termination features accessible only from one side of the film.

It will become apparent to those skilled in the art that this compliant dielectric film could be one of any polymer film or other surface capable of expanding and contracting.

In a third embodiment of the invention, the TSC's themselves are multi-layer flex circuits having 3 or more layers of TSC's which are interconnected by way of plated through-holes.

In a forth embodiment of the present invention the TSC's themselves utilize a surface mount technology to create TSC's with a compliant substrate. The present embodiment produces TSC's capable of reducing the negative effects of thermal expansion between selected materials.

In a fifth embodiment of the present invention the TSC's are polymer thick film flex circuits that incorporate a specially formulated conductive or resistive ink that is screen printed onto the flexible substrate to create the TCS patterns.

It will become apparent to those skilled in the art that these conductive and/or resistive inks can be any one of the many screenible types of ink that contain silver, carbon, or a silver/carbon mix to create the circuit patterns.

The width of the TCS mentioned in the five previous embodiments of the present invention, can vary from 0.005″ to 0.010″ depending on the needs of a particular thermography catheter, typical width and spacing being 0.015″.

### BRIEF DESCRIPTION OF THE DRAWINGS

The invention, together with further objects and advantages thereof, may best be understood by reference to the following description taken in conjunction with the accompanying drawings in which:

FIG. 1 illustrates a sectional view of the first step in constructing a flex circuit in accordance with the embodiments described in the present disclosure.

FIG. 2 illustrates a sectional view of the second step in constructing a flex circuit in accordance with the embodiments described in the present disclosure.

FIG. 3 illustrates a sectional view of the third step in constructing a flex circuit in accordance with the embodiments described in the present disclosure.

FIG. 4 illustrates a cross sectional view of a thermal mapping catheter with flex circuitry in accordance with the present disclosure.

FIG. 5 illustrates a cross sectional view of a thermal mapping catheter with flex circuitry taken at section 5-5 of FIG. 4 in accordance with the present disclosure.

FIG. 6 illustrates an overhead view of the flex circuit technology in accordance with a second embodiment in accordance with the present disclosure.

FIG. 7 illustrates a cross sectional view of the flex circuit technology taken at section 7-7 of FIG. 6 in accordance with a second embodiment of the present disclosure.

FIG. 8 illustrates an overhead view of the flex circuit technology in accordance with a third embodiment in accordance with the present disclosure.

FIG. 9 illustrates a cross sectional view of the flex circuit technology taken at section 9-9 of FIG. 8 in accordance with a third embodiment of the present disclosure.

FIG. 10 illustrates a cross sectional view of the flex circuit technology in accordance with a third embodiment in accordance with the present disclosure.

FIG. 11 diagrammatically illustrates the electrical circuitry of a third embodiment in accordance with the present disclosure.

FIG. 12 shows a perspective view of the expandable member of the present invention having a plurality of thermocouple sensors attached thereto.

FIG. 13 illustrates another embodiment of the present invention wherein the thermal sensor circuits comprise single sided flex circuits.

FIG. 14 illustrates another embodiment of the present invention wherein the thermal sensor circuits of the present invention comprise multiple layer flex circuits.

### DETAILED DESCRIPTION OF THE DRAWINGS

FIG. 1 is a cross sectional view of the first step in constructing a flex circuit 20 in accordance with the present invention. In FIG. 1 we see a typical configuration wherein a sheet of non-conductive compliant polymer approximately 3 mils thick forms a base layer 22. The base layer 22 is imprinted electrochemically with a series of conductive metallic strips 21a which form the upper layer of the flex circuit 20. The conductive metallic strips (CMS) 21a of the upper layer of the flex circuit 20 are approximately 5 mils thick and 5 mils wide. The CMS 21a are spaced 10 mils apart along the length of the base layer 22 creating a multiplicity of flexible circuits. It will become obvious to those skilled in the art that the thickness, width and spacing of the CMS 21a can be increased or decreased depending on the needs of a particular catheter.

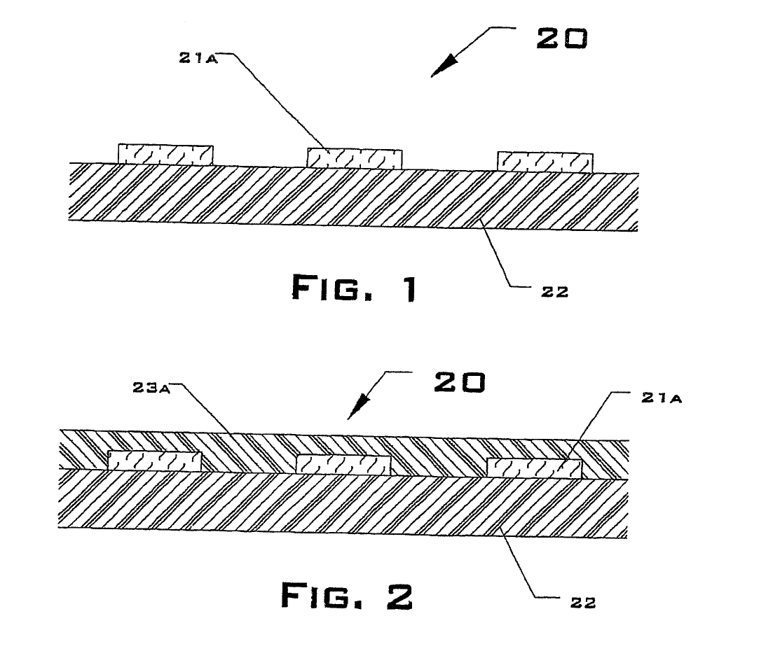

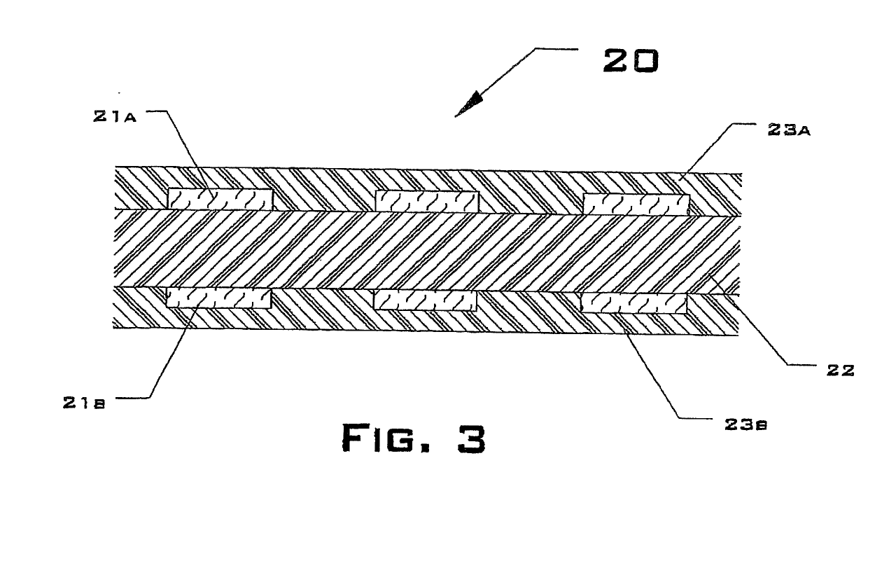

In FIG. 2 we see a cross section of the second step in constructing the flex circuit 20 in accordance with the present invention. Once the CMS 21a of the upper layer have been electrochemically imprinted onto the base layer 22 the flex strip is overcoated with a compliant non-conductive polymeric material 23a, to protect the CMS 21a from moisture. It will be obvious to those skilled in the art that this polymer over coating can be made from any of a number of commercially available compliant or non-compliant materials. In the example depicted in FIG. 2 the thickness of the resulting laminate is approximately 5 mils.

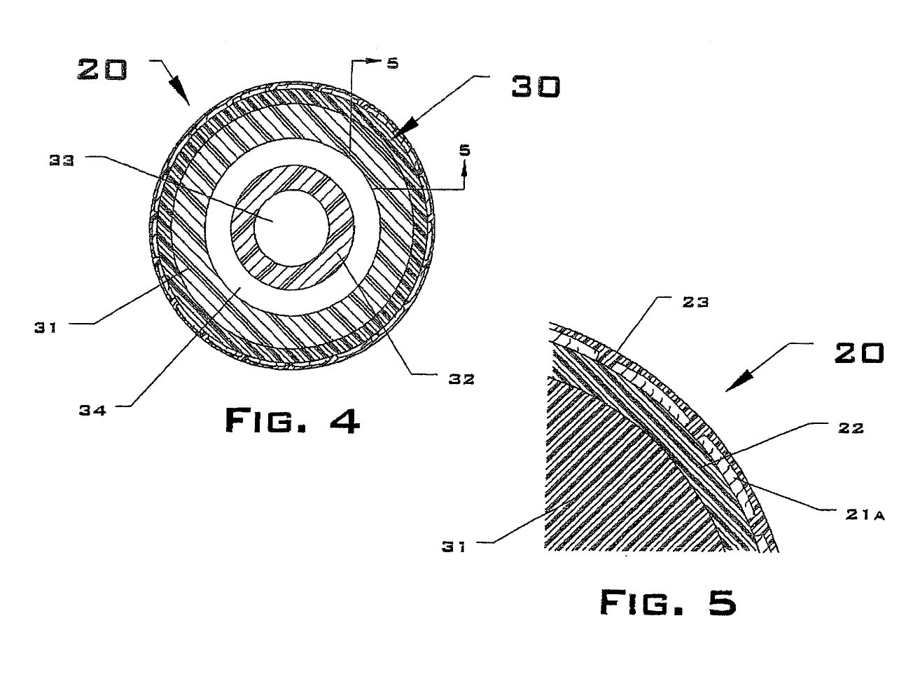

The completed flex circuit 20 is then wrapped around an intravascular catheter 30 and integrally bonded to its perimeter as shown in FIG. 4. The intravascular catheter 30, before the flex circuit 20 is attached, typically consists of two sizes of elongate tubular members, one placed within the other, so as to constitute an expansion lumen 34 and a guidewire lumen 33. However, it will become obvious to those skilled in the art that the flex circuit 20 can be attached to the perimeter of any kind of catheter.

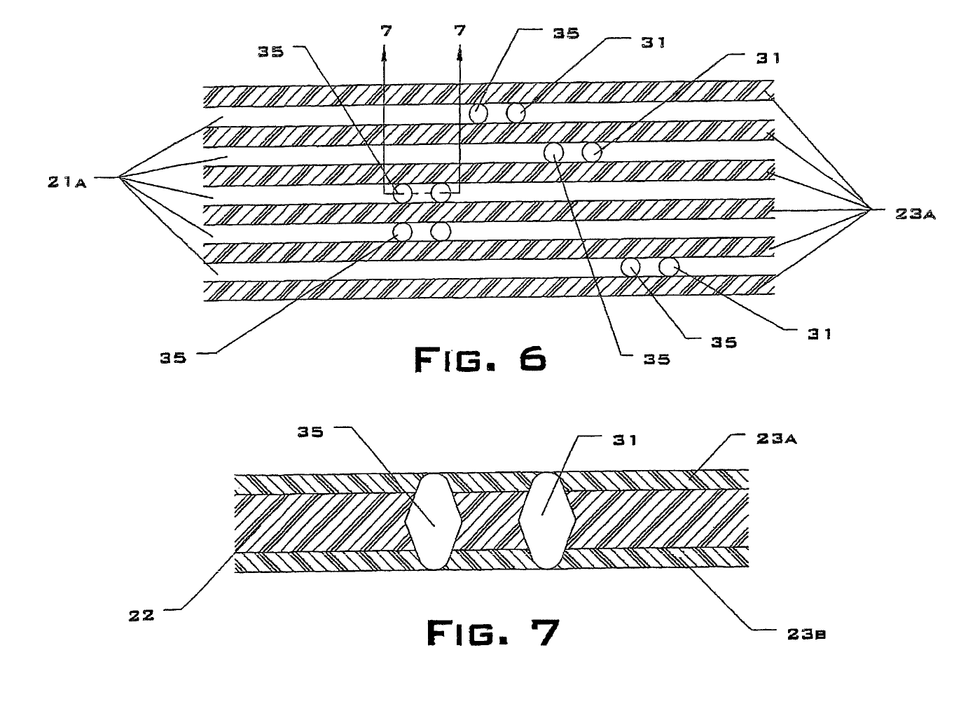

The catheter cross-section shown in FIG. 4 and FIG. 5 comprises the shaft portion of the catheter 30. The CMS 21a and 21b enable communication between the proximal hub portion (not shown) and the thermal sensors mounted on the expandable member.

Thermal Sensors

As mentioned previously, thermocouples are particularly advantageous because they can be fabricated directly onto the flex circuit 20. A thermocouple consists of a simple conductive junction between two dissimilar metals. The voltage generated at this junction is related to its temperature.

In FIG. 3 we see that the flex circuit 20 can be manufactured such that CMS 21a and 21b are on both sides of the base material 22. CMS 21a would be fabricated of material A and CMS 21b would be fabricated of material B where materials A and B define the thermocouple type. In FIG. 3 we see a cross sectional view of the third and final step taken to form the flex circuit 20.

FIGS. 6 and 7 show that a simple thermocouple may be formed anywhere along the flex circuit 20, by creating hole 35 through CMS 21a and CMS 21b directly where the thermocouple sensor is desired. A solder or weld joint is introduced into the hole 35 so as to electrically connect the CMS 21a and lower CMS 21b. If necessary, an additional hole 31 is made at a point further distal to the previous hole and filled with a non-conductive compliant polymer so as to prevent any electrical influences of the distal wires.

Serially Positioned Thermocouples to Obtain Temperature Difference

When two thermocouples are in series, the measured loop voltage is related to the temperature difference between the two thermocouples. A temperature difference between the lesion suspected to contain vulnerable plaque and a reference site proximal to the lesion may be more clinically meaningful than absolute temperature of the lesion. Thus in thermography applications, it may be desirable to place one thermocouple proximal to the expandable member in a presumed “normal” site (reference thermocouple) while one or more thermocouples mounted to the expandable member are placed over the suspected “abnormal” site (target site thermocouple).

The aorta is one example of a normal site that can be used in thermography applications, although any location in the vasculature, typically 5 centimeters away or greater from any portion of the target lesion is also suitable.

In one embodiment of this concept depicted schematically in FIG. 11 and further described below, a single reference thermocouple 36 may be electrically in series with a multiplicity of target site thermocouples 35. Both reference and target site thermocouples are created with the same pair of dissimilar materials A and B described earlier where the wires 21B between the reference thermocouple 36 and target site thermocouples 35 are made from material B and all remaining wires 21A in the series loop (wires not between thermocouples 35 and 36) are made from material A. The sensed voltage 40 is related to the temperature difference between the reference thermocouple 36 and each target site thermocouple 35. From a signal processing/engineering standpoint, this approach may lead to a more accurate result since the voltage difference between the two sensors is measured directly, as opposed to measuring two separate signals and then making a subtraction between them.

An illustration of the above concept is shown in FIGS. 8, 9, and 10. A single reference thermocouple 36 is created over any wire strip pair (21A and 21B) not already used for a target site thermocouple 35. The depicted example in FIG. 8 (top view showing “material A” side of the flex strip) shows the reference thermocouple 36 combined with 2 other target site thermocouples 35, although any number of target site thermocouples may also be used.

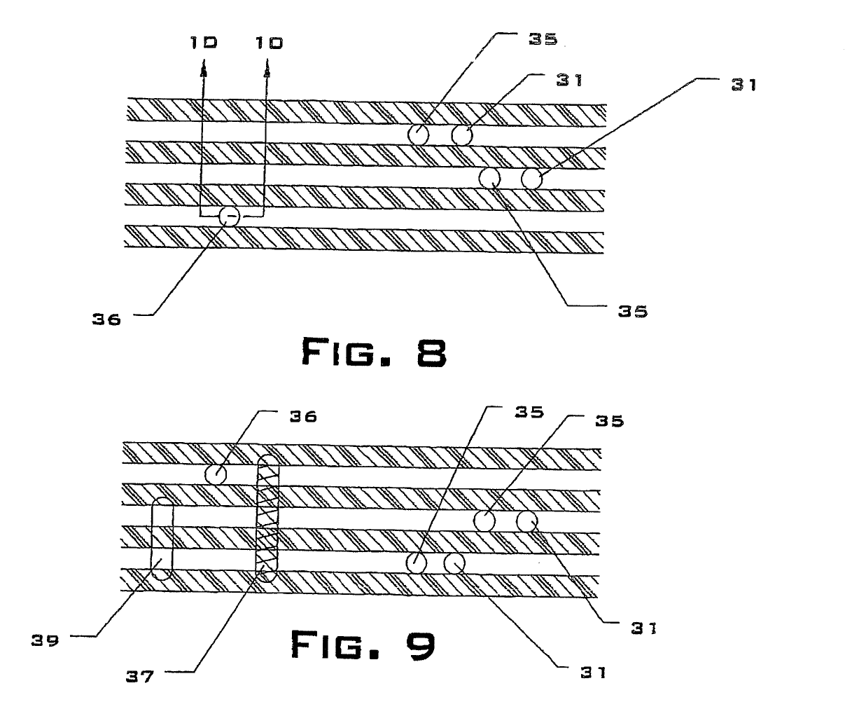

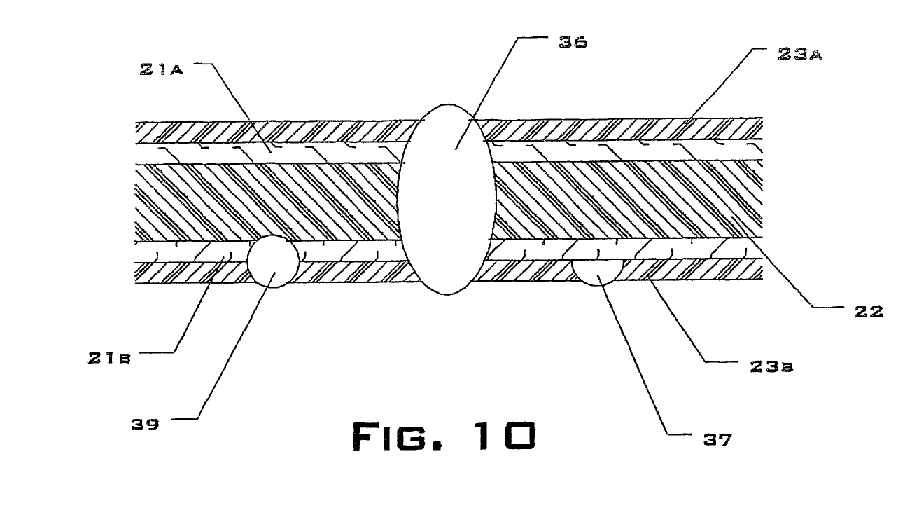

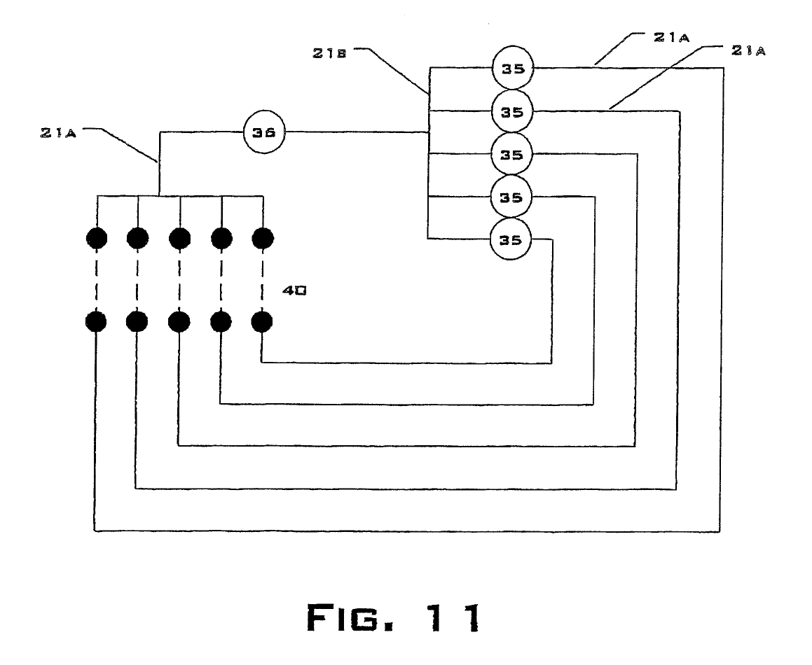

Reference thermocouple 36 is formed by first creating a hole all the way through upper CMS 21a and lower CMS 21b, and then forming a solder or weld joint through this hole as seen in FIG. 10. On the “material B” side of the flex strip shown in FIG. 9 (bottom view), wires 21B from all sensors (35 and 36) are electrically shorted together by stripping away sufficient material 23B such that wires 21B are exposed along a transverse path just distal to reference sensor 36, and then attaching a metallic strip 37 connecting all wires 21B along this path.

In one embodiment of this concept, wire 37 is electrochemically imprinted onto the flex strip using the same methods used to form CMS 21a and CMS 21b, although in principal any wire attachment method could be used. Also on the “material B” side of the flex strip, at a location just proximal to sensor 36, a transverse groove 39 is cut transverse to the flex strip such that wires 21B from all target site thermocouples 35 are cut. The wire 21B from reference sensor 36 is left uncut. This groove is filled with a non-conductive compliant polymer so as to prevent any electrical influences of proximal wires. Voltage 40 is sensed for each target site thermocouple 35 between the proximal terminating end of wire 21B for reference sensor 36 and the proximal terminating end of wire 21A for the target site thermocouple 35, at the proximal hub portion of the catheter (not shown).

Attachment of Flex Strip to an Expandable Member

As described earlier, electrical signals are communicated from thermal sensors mounted on the expandable member through a flex circuit 20 that is wrapped circumferentially around an expandable member. Those skilled in the art will appreciate the expandable member may comprise, for example, a balloon, an expandable wire structure, or an expandable wire basket as shown in U.S. patent application Ser. No. 09/340,089, filed on Jul. 25, 1999, naming Cassells et al. as first inventor, the disclosure of which is hereby incorporated by reference.

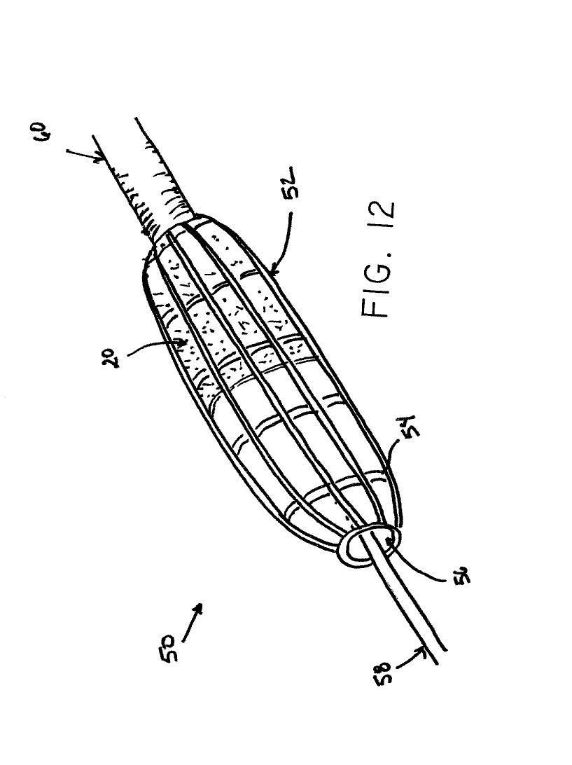

FIG. 12 shows the expandable member 50 of the present invention comprising an exterior portion 52 communicable with the vessel wall of a patient and capable of disposing at least one thermocouple 54 thereon, and an interior guidewire lumen 56 capable of receiving a guidewire 58. A flexible body member 60 may be in communication with the expandable member 50 to effectuate manipulation of the device through the patient's vessel. The expandable member 50 is capable of an unexpanded first diameter (not shown), and an expanded second diameter wherein the exterior portion 52 of the expandable member 50 is capable of engaging the vessel wall. An actuator (not shown) may be in communication with the expandable member and the operator may be used to effectuate expansion of the expandable member 50.

At the proximal end of the expandable member 50, it is convenient to have the flex circuit 20 split into separate “fibers” and be adhered to the exterior surface of the expandable member. The thermocouple sensors 54 are in communication with or have been fabricated into the flex circuit 20 at multiple desired positions in advance. In a preferred embodiment, it is desired to end up with thermocouple sensors 54 mounted on the expandable member 50 at regular axial spacings typically 1 cm apart, and at 4 circumferential locations 90 degrees apart. Those skilled in the art will appreciate that this spacing may vary with the specific needs of a particular catheter. In addition, the expandable member 50 may comprise a plurality of devices, including, for example, inflatable balloons and deployable wire structures.

The locations of the sensors as they are fabricated into the “flat” flex circuit 20 determine how they will be located when the strip is wrapped around the expandable member. Because each strand of thermocouple wire comes from a “strip”, it will tend to lie down in its intended position. The effect is like a partially peeled banana, where the peel, analogous to the flex circuit 20 is separated into multiple strands circumferentially. As a result, the strands may be pulled back to a desired axial position on the banana, analogous to the catheter shaft 30 while remaining connected to the banana: each strand of peel can be put back in its original location on the banana as long as its point of attachment is unbroken.

The adhering of the thermocouple wires to the expandable member will add mechanical stiffness to the expandable member in its length direction without affecting its circumferential stiffness. Thus, the expandable member will have less tendency to lengthen when expanded.

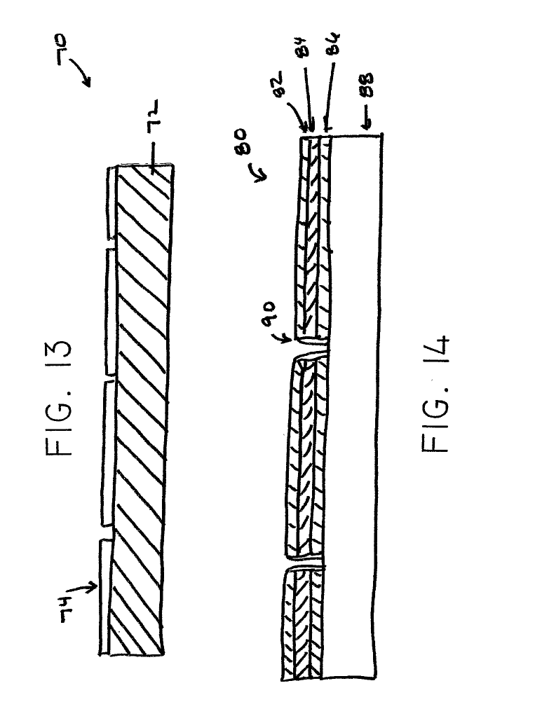

FIG. 13 shows a second embodiment of the invention wherein the TSC's themselves are single sided flex circuits. The single sided flex circuit 70 comprise a single conductor layer 72 of either metal or conductive polymer applied to a compliant dielectric film 74. As a result, the formed sensors are accessible only from one side of the film. Those skilled in the art will appreciate that this compliant dielectric film could be one of any polymer film or other surface capable of expanding and contracting.

FIG. 14 shows a third embodiment of the invention wherein the TSC's comprise multi-layer flex circuits having 3 or more layers. To form multi-layer flex circuits 80, three layers of flex circuits 82, 84, and 86 are applied to a dielectric substrate 88 and are interconnected through a series of plated through holes 90.

In yet another embodiment, the TSC's may comprise surface mounted electronic devices (commonly referred to SMTs) which provide the TSC's with a compliant substrate to reduce the effects of thermal expansion mismatches between the selected materials.

In another embodiment of the present invention, the TSC's may comprise polymer thick film flex circuits. The polymer thick film flex circuits incorporate a specially formulated conductive or resistive ink that is screen printed onto the flexible substrate to create the desired TCS patterns. Those skilled in the art will appreciate that the conductive and/or resistive inks can be any one of the many screenible types of ink that contain silver, carbon, or a silver/carbon mix to create the circuit patterns.

The width of the TCS mentioned in the five previous embodiments of the present invention can vary from 0.005″ to 0.010″ depending on the needs of a particular thermography catheter, typical width and spacing being 0.015″.

Although exemplary embodiments of the present invention have been described in some detail herein, the present examples and embodiments are to be considered as illustrative and not restrictive. The invention is not to be limited to the details given, but may be modified freely within the scope of the appended claims, including equivalent constructions.

What is claimed:

1. A device capable of measuring the temperature of a vessel wall of a patient, comprising:
an expandable member comprising an exterior portion and an interior portion;
said expandable member having first unexpanded diameter and a second expanded diameter, wherein said expandable member is capable of engaging a vessel wall when configured in said second diameter; and
a thermal sensor flex circuit in communication with said exterior portion of said expandable member, said thermal sensor flex circuit comprising at least one thermocouple.

2. The device of claim 1, wherein said thermal sensor flex circuit further comprises a plurality of thermocouples.

3. The device of claim 1, wherein said thermal sensor flex circuit is disposed on said exterior portion of said expandable member.

4. The device of claim 1, wherein said thermal sensor circuit comprises polyamide material electrochemically imprinted with at least one conductive strip.

5. The device of claim 4, wherein said polyamide is imprinted with a plurality of conductive strips.

6. The device of claim 1, wherein said thermal sensor flex circuits further comprise a single sided flex circuits, said single sided flex circuits having a single conductor layer applied to compliant dielectric material.

7. The device of claim 6, wherein said single conductor layer comprises a conductive metallic layer.

8. The device of claim 6, wherein said single conductor layer comprises a conductive polymer layer.

9. The device of claim 1, wherein said thermal sensor flex circuits further comprise a multiple layer flex circuit, said multiple layer flex circuits comprising at least three layers, said at least three layers interconnected through at least one through-hole.

10. The device of claim 9, wherein said at least one through-hole is plated with a conductive material.

11. The device of claim 1, wherein said thermal sensor flex circuits further comprise at least one surface mounted circuit.

12. The device of claim 11, wherein said at least one surface mounted circuit further comprises a compliant substrate capable of reducing a negative effect of thermal expansion.

13. The device of claim 1, wherein said thermal sensor circuit comprises a polyamide thick film, said polyamide thick film screen printed with a conductive ink.

14. The device of claim 1, wherein said thermal sensor circuit comprises a polyamide thick film, said polyamide thick film screen printed with a electrically resistive ink.

15. The device of claim 1, wherein said expandable member is a balloon.

16. The device of claim 1, wherein said expandable member is a deployable wire structure.

17. The device of claim 1, wherein said expandable member further comprises an actuator, said actuator capable of being actuated by a user.

18. A device capable of measuring the temperature of a vessel wall of a patient, comprising:
an expandable member comprising an exterior portion and an interior portion;
said expandable member having first unexpanded diameter and a second expanded diameter, wherein said expandable member is capable of engaging a vessel wall when configured in said second diameter; and
a single sided thermal sensor flex circuit in communication with said exterior portion of said expandable member, said single sided thermal sensor flex circuit comprising a single conductor layer applied to compliant dielectric material and at least one thermocouple.

19. A device capable of measuring the temperature of a vessel wall of a patient, comprising:
an expandable member comprising an exterior portion and an interior portion;
said expandable member having first unexpanded diameter and a second expanded diameter, wherein said expandable member is capable of engaging a vessel wall when configured in said second diameter; and
a multiple layer thermal sensor flex circuit in communication with said exterior portion of said expandable member, said multiple layer thermal sensor flex circuit comprising at least three layers and at least one thermocouple, said at least three layers interconnected through at least one through-hole.
